(19) United States
(12) Patent Application Publication
(10) Pub. No.: US 2016/0074310 A1
(43) Pub. Date: Mar. 17, 2016

(54) Composition Containing Oil Bodies, Fatty Acids, Amino Acid Surfactants And N-Methyl-N-Acylglucamines

(71) Applicant: CLARIANT INTERNATIONAL LTD, Muttenz (CH)

(72) Inventors: Peter KLUG, Grossostheim (DE); Carina MILDNER, Frankfurt am Main (DE); Martin BAUER, Frankfurt am Main (DE)

(73) Assignee: CLARIANT INTERNATIONAL LTD., Muttenz (CH)

(21) Appl. No.: 14/785,599

(22) PCT Filed: Apr. 16, 2014
(86) PCT No.: PCT/EP2014/001022

§ 371 Date: Oct. 19, 2015

(30) Foreign Application Priority Data
Apr. 20, 2013 (DE) 10 2013 006 879.7

(51) Int. Cl. A61K 8/92; A61K 8/44; A61K 8/41; A61K 8/36; A61K 8/37; A61K 8/46; A61Q 19/10; A61K 8/60

(52) U.S. Cl. CPC A61K 8/92; A61Q 19/10; A61K 8/44; A61K 8/60; A61K 8/361; A61K 8/37; A61K 8/466; A61K 8/416

(57) ABSTRACT

The invention relates to compositions containing: (A) at least one N-acyl-amino acid surfactant as component A, (B) at least one N-methyl-N-acylglucamine as component B, (C) at least one C₈-C₂₂ fatty acid or a fatty acid salt as component C, (D) at least one oil body as component D, (E) optionally at least one acylisethionate as component E, (F) optionally at least one sulfonated anionic surfactant as component F, (G) optionally at least one betaine surfactant as component G, (H) optionally at least one additive as component H, and (I) water. The compositions are suitable for cosmetic, dermatological, and pharmaceutical applications.

The invention relates to a composition comprising oily substances, fatty acids, amino acid surfactants and N-methyl-N-acylglucamines. The invention relates further to the use of the composition for the treatment or care of the skin or hair, for example as a shampoo, facial cleanser, liquid cleaner or shower gel.

The production of liquid products in the cosmetics and detergents sector is increasing constantly. Especially in the field of body cleansing agents, it is liquid compositions such as hair shampoos, foam baths and shower gels which have increasingly gained importance in recent years.

As well as comprising water and surfactants, such compositions frequently comprise an oily substance, for example natural oils or paraffins. In combination with fatty acids, a pleasant feel on the skin is thereby produced. However, oily substances, in particular in combination with fatty acids, represent a challenge for the development of a suitable surfactant system.

A requirement for a good surfactant formulation is good storage stability. The composition must not become cloudy or form sediment in the case of fluctuations in temperature and should have a viscosity which can be adapted to the particular intended use. The viscosity profile of the formulation is additionally to be constant over a wide temperature range, so that the formulation can be handled regardless of the ambient temperature. Accordingly, the viscosity is a quality criterion. The degree of viscosity depends on the surfactant system, the electrolyte addition and also on the content of oily substances in the surfactant formulation. If oily substances are added to formulations comprising surfactants, a drastic fall in the viscosity is generally seen. Emulsions form, which in practice have to be thickened with thickening polymers, such as, for example, polyacrylates, xanthan gum or starch derivatives such as sodium hydroxypropyl starch phosphate, in order to achieve sufficient stability.

Accordingly, the object of the invention is to provide improved oil-containing compositions which produce more stable emulsions, reduce the need for thickening polymers and permit more temperature-stable formulations.

The use of N-polyhydroxyalkyl fatty acid amides in cosmetic compositions and detergents is known.

N-Methyl-N-acylglucamines having C₁₂- and C₁₄-acyl groups and fatty acid ether sulfates or fatty acid sulfates are described in WO 92/06158 and WO 92/06162 for use in the detergents and cleaning agents.

WO 98/56496 relates to a surfactant composition having improved foam stability. The surfactant composition comprises: (a) from approximately 1 to approximately 40% by weight of a sugar surfactant; (b) from approximately 1 to approximately 40% by weight of an anionic surfactant; (c) from approximately 0.11 to approximately 10% by weight of an amphoacetate; and (d) water, wherein the amounts by weight are based on the weight of the composition.

In EP-A 0 285 768, the use of N-polyhydroxyalkyl fatty acid amides as thickening agents for liquid aqueous surfactant systems is described.

It cannot be inferred from any of the mentioned documents that N-methyl-N-acylglucamines in combination with specific further surfactants are suitable in particular for use in compositions comprising oily substances and fatty acids.

It has now been found that compositions which, as well as comprising an oily substance and fatty acid(s), comprise a combination of an amino acid surfactant and a glucamide achieve the mentioned object.

Accordingly, there is provided a composition comprising:

(A) at least one N-acyl-amino acid surfactant as component A,

(B) at least one N-methyl-N-acylglucamine as component B,

(C) at least one C₈-C₂₂-fatty acid or a fatty acid salt as component C,

(D) at least one oily substance as component D,

(E) optionally at least one acyl isethionate as component E,

(F) optionally at least one anionic, sulfonated surfactant as component F,

(G) optionally at least one betaine surfactant as component G,

(H) optionally one or more additives as component H, and

(I) water.

The composition according to the invention advantageously exhibits improved emulsion stability and improved viscosity behavior at high temperature, and thus improved storage stability.

Preference is given according to the invention to a composition comprising:

(A) at least one N-acyl-amino acid surfactant as component A,

(B) at least one N-methyl-N-acylglucamine as component B,

(C) at least one C₁₂-C₂₂ fatty acid or a fatty acid salt as component C,

(D) at least one oily substance as component D,

(E) optionally at least one acyl isethionate as component E,

(F) optionally one or more anionic, sulfonated surfactants as component F,

(G) optionally one or more betaine surfactants as component G,

(H) optionally one or more additives as component H, and

(I) water.

Preference is also given to a composition which, as well as comprising components A, B, C and D, comprises component F. Preference is also given to a composition which, as well as comprising components A, B, C and D, comprises component G. Preference is also given to a composition which, as well as comprising components A, B, C and D, comprises component H.

Preference is also given to a composition which, as well as comprising components A, B, C and D, comprises components E and F. Preference is also given to a composition which, as well as comprising components A, B, C and D, comprises components E and G. Preference is also given to a composition which, as well as comprising components A, B, C and D, comprises components E and H.

Preference is also given to a composition which, as well as comprising components A, B, C and D, comprises components F and G. Preference is also given to a composition which, as well as comprising components A, B, C and D, comprises components F and H. Preference is also given to a composition which, as well as comprising components A, B, C and D, comprises components G and H.

Preference is also given to a composition which, as well as comprising components A, B, C and D, comprises components E, F and G.

Preference is also given to a composition which, as well as comprising components A, B, C and D, comprises components E, F and H. Preference is also given to a composition which, as well as comprising components A, B, C and D, comprises components E, G and H.

Preference is also given to a composition which, as well as comprising components A, B, C and D, comprises components F, G and H. Preference is also given to a composition which, as well as comprising components A, B, C and D, comprises components E, F, G and H.

Preference is further given to a composition comprising:

(A) from 0.5 to 10% by weight of component A,

(B) from 0.5 to 10% by weight of component B,

(C) from 0.5 to 5.0% by weight of component C,

(D) from 0.5 to 8.0% by weight of component D,

(E) from 0 to 8.0% by weight of component E,

(F) from 0 to 10% by weight of component F,

(G) from 0 to 8% by weight of component G,

(H) from 0 to 15% by weight of one or more additives H,

(I) from 74 to 98% by weight water,

wherein the sum of components A to I is 100% by weight.

Preference is further given to compositions comprising:

(A) from 1 to 6.0% by weight of component A, preferably from 2.0 to 4.0% by weight,

(B) from 1 to 5.0% by weight of component B, preferably from 2.0 to 4.0% by weight,

(C) from 1 to 4.0% by weight of component C, preferably from 1.5 to 3.0% by weight,

(D) from 1 to 5.0% by weight of component D, preferably from 1.5 to 3.0% by weight,

(E) 0 or from 0.5 to 2.0% by weight of component E,

(F) 0 or from 1 to 5% by weight of component F, preferably from 0.5 to 3.0% by weight,

(G) 0 or from 0.5 to 3% by weight of component G,

(H) 0 or from 5 to 10% by weight of one or more additives H, and

(I) from 40 to 98 or from 40 to 89% by weight water, wherein the sum of components A to I is 100% by weight.

The value characterized as “or” denotes the preferred lower limit if the component is present. In the case of water (I), the value “or” denotes the limits if all the components are present.

All the mentioned compositions preferably consist of components (A) to (I).

(A)

Within the scope of a preferred embodiment, the amino acid radical of component (A) is selected from the group consisting of proteinogenic amino acids, their N-alkylated derivatives, mixtures thereof, taurine and N-methyltaurine.

Within the scope of a preferred embodiment, component A is selected from the group consisting of acyl glycinate, acyl aspartate, acyl glutamate, acyl sarcosinate or mixtures thereof.

Preference is given to acyl glycinates, acyl alaninates, acyl aspartates, acyl glutamates and acyl sarcosinates, in particular sodium cocoyl glycinate, potassium cocoyl glycinate, sodium lauroyl glycinate, potassium lauroyl glycinate, sodium cocoyl glutamate, sodium lauroyl glutamate, sodium cocoyl aspartate, sodium lauroyl aspartate and sodium lauroyl sarcosinate.

Within the scope of a preferred embodiment, component A consists of at least one C₈-C₂₂-acylated amino acid, in particular N-alkylated derivatives thereof. Preference is given to the corresponding lauroyl or cocoyl derivatives of the amino acids.

Sodium acyl glycinate and potassium acyl glycinate are particularly preferred. Sodium cocoyl glycinate and sodium lauroyl glycinate are also particularly preferred.

(B)

Further terms for N-methyl-N-acylglucamine are N-methyl-N-1-deoxysorbitol fatty acid amide, N-acyl-N-methyl-glucamine, glucamide or N-methyl-N-alkylglucamide.

As a preferred embodiment there are used as component (B) N-methyl-N-acylglucamines and mixtures thereof (also known as N-methyl-N-1-deoxysorbityl fatty acid amides) which carry a saturated or unsaturated, straight-chained or branched C₈-C₂₂-acyl radical, preferably a straight-chained, saturated or unsaturated C₁₂-C₁₈-acyl radical. N-Methyl-N-acylglucamine thereby corresponds to formula (I), wherein Ra is a C₇-C₂₁-hydrocarbon radical.

Particularly preferred as component (B) are N-methyl-N-acylglucamines of formula (I) wherein the acyl radical RaCO is derived from octanoic acid, decanoic acid, lauric acid, myristic acid, palmitic acid, stearic acid, oleic acid, linoleic acid or linolenic acid:

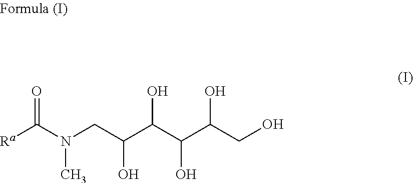

Particularly preferably, the proportion of N-methyl-N-acylglucamines containing a C₁₂-, C₁₄- or an unsaturated C₁₈-acyl group is at least 70% by weight and the proportion of N-methyl-N-acylglucamines containing an acyl group <C₁₂ is less than 3% by weight.

Most particularly preferably, the proportion of N-methyl-N-acylglucamines containing a C₁₂-C₁₄- or an unsaturated C₁₈-acyl group is at least 80% by weight and the proportion of N-methyl-N-acylglucamines containing an acyl group <C₁₂ is less than 2% by weight.

In a further embodiment, the proportion of N-methyl-N-acylglucamines containing a C₁₂- or a C₁₄-acyl group is at least 90% by weight and the proportion of N-methyl-N-acylglucamines containing an acyl group <C₁₂ is less than 2% by weight.

The N-methyl-N-acylglucamines used according to the invention as thickeners additionally comprise small proportions of N-methyl-N-acyl-glucamines derived from short-chained and/or long-chained fatty acids, in particular those which contain C₁-C₄-acyl, C₆-, C₈-, C₁₀-, C₁₆-, C₁₈- and/or C₂₀-acyl groups.

Within the scope of a further preferred embodiment, component B consists of a mixture of N-methyl-N-acylglucamines wherein at least 80% by weight of the N-methyl-N-acylglucamines have a saturated or unsaturated C₁₆- or C₁₈-acyl radical. For example, there can be present in the mixture also N-methyl-N-acylglucamines that have a C₁₄-acyl radical but wherein at least 80% by weight of the N-methyl-N-acylglucamines have a saturated or unsaturated C₁₆- to C₁₈-acyl radical. Component C preferably consists of a mixture of N-methyl-N-acylglucamines wherein at least 90% by weight of the N-methyl-N-acylglucamines have a saturated or unsaturated C₁₆- or C₁₈-acyl radical.

(C)

The fatty acids of component (C) are preferably natural fatty acids, preferably having from 8 to 22 carbon atoms, for example octanoic acid, decanoic acid, lauric acid, myristic acid, palmitic acid, stearic acid and behenic acid and mixtures thereof, as well as their alkali salts such as sodium stearate, sodium palmitate, sodium laurate, sodium myristate, sodium behenate, potassium stearate, potassium palmitate, sodium myristate, hydroxy fatty acids, for example 12-hydroxystearic acid or 16-hydroxyhexadecanoic acid and their salts. Preference is given to lauric acid, stearic acid and mixtures and salts thereof. The fatty acids are generally used to impart to the composition a lipid-replenishing and caring feel on the skin.

(D)

The oily substances as component (D) can advantageously be selected from the groups of the natural and synthetic fatty substances, preferably triglycerides, esters of fatty acids with alcohols having a low C number, for example with isopropanol, propylene glycol or glycerol, or esters of fatty alcohols with alkanoic acids having a low C number or with fatty acids or from the group of the alkyl benzoates, as well as natural or synthetic hydrocarbon oils and silicone oils.

Triglyceride oils such as sunflower oil and soybean oil are particularly preferred; petrolatum (Vaseline) is likewise particularly preferred.

There come into consideration preferably triglycerides of linear or branched, saturated or unsaturated, optionally hydroxylated C₈-C₃₀-fatty acids, in particular vegetable oils, such as sunflower oil, corn oil, soybean oil, rice oil, jojoba oil, babassu oil, pumpkin oil, grapeseed oil, sesame oil, walnut oil, apricot oil, orange oil, wheatgerm oil, peach kernel oil, macadamia oil, avocado oil, sweet almond oil, cuckoo flower oil, castor oil, olive oil, groundnut oil, rape oil and coconut oil, as well as synthetic triglyceride oils, for example the commercial product Myritol® 318. Hardened triglycerides are also preferred according to the invention. Oils of animal origin, for example beef tallow, perhydrosqualene, lanolin, can also be used.

Preferred oily substances according to the invention are also the benzoic acid esters of linear or branched C₈-C₂₂-alkanols, for example the commercial products Finsoly® SB (isostearyl benzoate), Fins® Iv® TN (C₁₂-C₁₅-alkyl benzoate) and Fins® Iv® EB (ethylhexyl benzoate).

A further class of oily substances that are preferred according to the invention are dialkyl ethers having a total of from 12 to 36 carbon atoms, in particular having from 12 to 24 carbon atoms, such as, for example, di-n-octyl ether (Cetiol® OE), di-n-nonyl ether, di-n-decyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, di-3-ethyldecyl ether, tert-butyl n-octyl ether, isopentyl n-octyl ether and 2-methylpentyl n-octyl ether as well as di-tert-butyl ether and diisopentyl ether.

There come into consideration also branched saturated or unsaturated fatty alcohols having from 6 to 30 carbon atoms, for example isostearyl alcohol, as well as Guerbet alcohols.

A further class of oily substances that are preferred according to the invention are dicarboxylic acid esters of linear or branched C₂-C₁₀-alkanols, such as di-n-butyl adipate, (Cetiol® B), di-(2-ethylhexyl)adipate and di-(2-ethylhexyl)succinate, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate and neopentyl glycol dicaprylate, as well as diisotridecyl acetate.

Preferred oily substances are likewise symmetrical, non-symmetrical or cyclic esters of carbonic acid with fatty alcohols, glycerol carbonate or dicaprylyl carbonate (Cetiol® CC).

A further class of oily substances that are preferred according to the invention are the esters of dimers of unsaturated C₁₂-C₂₂-fatty acids (dimer fatty acids) with monovalent linear, branched or cyclic C₂-C₁₈-alkanols or with polyvalent linear or branched C₂-C₆-alkanols.

A further class of oily substances that are preferred according to the invention are hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, as well as hexadecane, isohexadecane, paraffin oils, isoparaffin oils, for example the commercial products of the Permethyl® series, squalane, squalene, and alicyclic hydrocarbons, for example the commercial product 1,3-di-(2-ethyl-hexyl)-cyclohexane (Cetiol® S), ozokerite and ceresin.

(E)

Within the scope of a preferred embodiment, the composition according to the invention comprises at least one acyl isethionate of formula (II) as component (E):

R—CO—O—CHR¹—CHR²—SO₃X (II)

wherein
R denotes the alkyl radical of a C₈-C₁₈-fatty acid,
R¹ and R² independently of one another denote H or CH₃, preferably H, and
X is a cation, preferably an alkali metal cation, in particular Na.

These include acyl isethionates and methyl-acyl isethionates having a C₈-C₁₈-acyl radical and mixtures thereof, preferably sodium salts thereof. Sodium cocoylisethionate and sodium lauroylisethionate are particularly preferred.

Compositions that also comprise component (E) exhibit particularly high stability.

(F)

Within the scope of a preferred embodiment, the composition according to the invention comprises at least one anionic, sulfonated surfactant as component (F).

In a preferred embodiment, component F is selected from one or more compound(s) of the general formula (III)

R¹SO₃⁻M⁺ (III)

wherein
R¹ represents alkyl, cycloalkyl, aralkyl, aryl, alkoxy, alkoxyalkyl and heterocyclyl, and
M⁺ is an alkali metal, alkaline earth metal or a substituted or unsubstituted ammonium ion, or
of the general formula (IV)

R¹SO₄⁻M⁺ (IV)
wherein
R¹ represents alkyl, cycloalkyl, aralkyl, aryl, alkoxy, alkoxyalkyl and heterocyclyl, and
M⁺ is an alkali metal, alkaline earth metal or a substituted or unsubstituted ammonium ion.

“Alkyl” denotes a saturated aliphatic hydrocarbon group which can be straight-chained or branched and can have from 1 to 20 carbon atoms in the chain. Preferred alkyl groups can be straight-chained or branched and have from 1 to 10 carbon atoms in the chain. Branched means that a lower alkyl group, such as methyl, ethyl or propyl, is attached to a linear alkyl chain. Alkyl is, for example, methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2, 3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl.

“Cycloalkyl” denotes an aliphatic ring which has from 3 to 10 carbon atoms in the ring. Preferred cycloalkyl groups have from 4 to 7 carbon atoms in the ring.

“Aryl” denotes phenyl or naphthyl.

“Aralkyl” denotes an alkyl group which is substituted by an aryl radical.

“Substituted aralkyl” and “substituted aryl” mean that the aryl group or the aryl group of the aralkyl group is substituted by one or more substituents selected from alkyl, alkoxy, nitro, carboalkoxy, cyano, halo, alkylmercaptyl, trihaloalkyl or carboxyalkyl.

“Alkoxy” denotes an alkoxy-O group in which “alkyl” has the meaning described above. Lower alkoxy groups are preferred. Examples are methoxy, ethoxy, n-propoxy, isopropoxy and n-butoxy.

“Lower alkyl” denotes an alkyl group which has from 1 to 7 carbon atoms. “Alkoxyalkyl” denotes an alkyl group as described above which is substituted by an alkoxy group as described above. Accordingly, the term alkoxyalkyl can be understood as meaning a polyether.

“Heterocyclyl” denotes a 4- to 10-membered ring structure in which one or more ring atoms are other than carbon, for example N, O or S. Heterocyclyl can be aromatic or non-aromatic, that is to say it can be saturated, partially unsaturated or completely unsaturated.

Within the scope of a preferred embodiment, the anionic surfactant of component A is an alkyl sulfate or an alkyl ether sulfate. Sodium lauryl sulfate, sodium laureth sulfate (1 or 2 EO units) or mixtures thereof are particularly preferred.

In a further preferred embodiment, the composition according to the invention does not comprise a component (F).

(G)

Within the scope of a preferred embodiment, the composition according to the invention comprises at least one alkyl betaine and/or at least one alkylamido betaine as component (G).

Examples of suitable alkyl betaines are the carboxyalkylation products of secondary and in particular tertiary amines of formula (V)

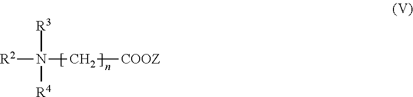

wherein
R² represents alkyl and/or alkenyl radicals having from 6 to 22 carbon atoms,
R³ represents hydrogen or alkyl radicals having from 1 to 4 carbon atoms, R⁴ represents hydrogen or alkyl radicals having from 1 to 4 carbon atoms,
n represents numbers from 1 to 6, and
Z represents an alkali and/or alkaline earth metal or ammonium.

Typical examples are the carboxymethylation products of hexylmethylamine, hexyldimethylamine, octyldimethylamine, decyldimethyl-amine, dodecylmethylamine, dodecyldimethylamine, dodecylethylmethyl-amine, C_{12/14}-coco alkyldimethylamine, myristyldimethylamine, cetyl-dimethylamine, stearyldimethylamine, stearylethylmethylamine, oleyl-dimethylamine, C₁₆-C₁₈-tallow alkyldimethylamine, and commercial mixtures thereof.

Examples of suitable alkylamido betaines are carboxyalkylation products of amidoamines. Particularly suitable are amidopropyl betaines of formula (VI)

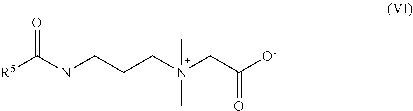

wherein R⁵ is a linear or branched saturated C₇-C₂₁-alkyl group or a linear or branched mono- or poly-unsaturated C₇-C₂₁-alkenyl group.

Preferred betaine surfactants are amidopropyl betaines such as cocoamidopropyl betaine (R⁵CO is the fatty acid radical of coconut oil, chain length C₈-C₁₈) and alkyl betaines such as coco-betaine (R² is the alkyl radical of coconut oil, chain length C₈-C₁₈) or lauryl betaine (R² is an alkyl radical of chain length C₁₂ and C₁₄).

(H)

Within the scope of a preferred embodiment, the composition according to the invention comprises one or more additives, preferably from the group consisting of preservatives, fragrances, dyes, further surfactants which do not fall under the definition of components A to G, solvents, cationic polymers, film-forming agents, thickeners and gelling agents, superfatting agents, antimicrobial and biogenic active ingredients, moisture-donating substances, stabilizers, acids, lyes, activity enhancers and mixtures thereof, preferably in amounts of from 0.1 to 10.0% by weight, particularly preferably from 3.0 to 15.0% by weight, and in particular from 5.0 to 10.0% by weight.

Suitable preservatives are preservatives listed in the relevant annex to the European cosmetic products legislation, for example phenoxyethanol, benzyl alcohol, parabens, benzoic acid and sorbic acid; there are particularly suitable, for example, 1,3-bis(hydroxymethyl)-5,5-dimethylimidazolidine-2,4-dione (Nipaguard® DMDMH), piroctone olamine, methylisothiazolinone or mixtures thereof, preferably piroctone olamine and/or methylisothiazolinone.

As fragrances or perfumes or oils there can be used individual fragrance compounds, for example synthetic products of the ester, ether, aldehyde, ketone, alcohol and hydrocarbon type. Examples of fragrance compounds of the ester type are benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allylcyclohexyl propionate, styrallyl propionate and benzyl salicylate. The ethers include, for example, benzyl ethyl ether; the aldehydes include, for example, linear alkanals having from 8 to 18 carbon atoms, citral, citronellal, citronellyloxy acetaldehyde, cyclamen aldehyde, hydroxycitronellal, lilial and bourgeonal; the ketones include, for example, ionones, alpha-isomethylionone and methyl cedryl ketone; the alcohols include anethol, citronellol, eugenol, geranion, linalool, phenylethyl alcohol and terpineol; the hydrocarbons include mainly terpenes and balsams. Preference is given to the use of mixtures of different fragrances, which together produce a pleasant note.

Perfume oils can also comprise natural fragrance mixtures, as are obtainable from plant or animal sources, for example pine oil, citrus oil, jasmine oil, lily oil, rose oil or ylang-ylang oil. Ethereal oils of low volatility, which are mostly used as flavor components, are also suitable as perfume oils, for example salvia oil, chamomile oil, carnation oil, melissa oil, mint oil, cinnamon leaf oil, linden blossom oil, juniper berry oil, vetiver oil, olibanum oil, galbanum oil and ladanum oil.

There are suitable as dyes in principle any dyes which are permitted for cosmetics use; such dyes are listed in the corresponding annexes of the European cosmetics legislation.

Further surfactants which do not fall under the definition of (A) to (G) can in principle be any anionic, cationic or amphoteric surfactants which are suitable for cosmetics. Preference is given to:

Ethoxylated and propoxylated fatty alcohols, ethoxylated and propoxylated triglycerides such as PEG-40 hydrogenated castor oil or fatty acid esters, ether carboxylates, alkyl polyglucosides, olefin sulfonates, sec. alkyl sulfonates and taurates.

A solvent within the scope of the present invention is understood as being preferably protic solvents such as water, C₁-C₈-alcohols, in particular C₁-C₆-alcohols, ethylene glycol, diethylene glycol, triethylene glycol or mixtures thereof, wherein water and/or ethanol or water and/or methanol are particularly preferred. Of the C₁-C₆-alcohols, methanol, ethanol, isopropanol, n-butanol or sec-butanol are preferred.

Suitable cationic polymers are those known by the INCl name “polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, as well as polyquaternium 37& mineral oil & PPG trideceth (Salcare SC95), PVP-dimethylaminoethyl methacrylate copolymer, guar hydroxypropyltriammonium chloride, as well as calcium alginate and ammonium alginate. There can further be used cationic cellulose derivatives; cationic starches; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicone; copolymers of adipic acid and dimethylam inohydroxy-propyldiethylenetriamine; polyaminopolyamide and cationic chitin derivatives, such as, for example, chitosan.

The compositions according to the invention can further comprise film-forming agents, which are selected, according to the intended use, from salts of phenylbenzimidazolesulfonic acid, water-soluble polyurethanes, for example Cio-polycarbamyl polyglyceryl ester; polyvinyl alcohol, polyvinylpyrrolidone copolymers such as PVP/hexanedecene or PVP/eicosene copolymer, for example vinylpyrrolidone/vinyl acetate copolymer, water-soluble acrylic acid polymers/copolymers or esters or salts thereof, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropyl-cellulose, water-soluble quaterniums, polyquaterniums, carboxyvinyl polymers, such as carbomers and salts thereof, polysaccharides, for example polydextrose and glucan, vinyl acetate/crotonate, for example obtainable under the trade name Aristoflex® A 60 (Clariant), as well as polymeric amine oxides, for example representatives obtainable under the trade names Diaformer Z-711, 712, 731, 751.

The desired viscosity of the compositions can be adjusted (increased or lowered) by adding thickeners and gelling agents. There come into consideration preferably cellulose ethers and other cellulose derivatives (for example carboxymethylcellulose, hydroxyethylcellulose), gelatin, starch and starch derivatives such as hydroxypropyl starch phosphate or sodium hydroxypropyl starch phosphate, sodium alginates, fatty acid polyethylene glycol esters, agar-agar, tragacanth or dextrin derivatives, in particular dextrin esters. Also suitable are fatty acid amides; fatty acid alkanolamides; dibenzalsorbitol and alcohol-soluble polyamides and polyacrylamides or mixtures thereof. There can further be used crosslinked and uncrosslinked polyacrylates such as carbomers, sodium polyacrylates or sulfonic-acid-containing polymers such as ammonium acryloyldimethyltaurateNP copolymer.

There can be used as superfatting agents preferably lanolin and lecithin, non-ethoxylated and polyethoxylated or acylated lanolin and lecithin derivatives, polyol fatty acid esters, mono-, di- and tri-glycerides and/or fatty acid alkanolamides, as well as ethoxylated triglycerides such as PEG-7 glyceryl cocoate or mixtures of glyceryl oleate with alkyl polyglucosides.

There are used as antimicrobial active ingredients cetyltrimethylammonium chloride, cetylpyridinium chloride, benzethonium chloride, diisobutylethoxy-ethyldimethylbenzylammonium chloride, sodium N-lauryl sarcosinate, sodium N-palmethyl sarcosinate, lauroyl sarcosine, N-myristoylglycine, potassium N-lauryl sarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctose, in particular zinc salts, pyrithiones and heavy metal salts thereof, in particular zinc pyrithione, zinc phenolsulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and octopirox, iodopropynyl butylcarbamate, methyl-chloroisothiazolinone, methylisothiazolinone, methyldibromo glutaronitrile, AgCl, chloroxylenol, sodium salt of diethylhexyl sulfosuccinate, sodium benzoate, as well as phenoxyethanol, benzyl alcohol, phenoxyisopropanol, parabens, preferably butyl-, ethyl-, methyl- and propyl-paraben, and sodium salts thereof, pentanediol, 1,2-octanediol, 2-bromo-2-nitropropane-1,3-diol, ethylhexylglycerol, benzyl alcohol, sorbic acid, benzoic acid, lactic acid, imidazolidinylurea, diazolidinylurea, dimethyloldimethylhydantoin (DMDMH), sodium salt of hydroxymethyl glycinate, hydroxyethylglycine of sorbic acid, and combinations of these active substances.

The compositions according to the invention can further comprise biogenic active ingredients selected from plant extracts, such as, for example, aloe vera, as well as local anesthetics, antibiotics, antiphlogistics, antiallergics, corticosteroids, sebostatic agents, Bisabolol®, Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as the sodium salt of the monophosphoric acid ester of ascorbic acid or as the magnesium salt of the phosphoric acid ester of ascorbic acid, tocopherol and tocopherol acetate, as well as vitamin E and/or derivatives thereof.

There are available as the moisture-donating substance, for example, isopropyl palmitate, glycerol, diglycerol and/or sorbitol. Glycerol is particularly preferred.

As acids or lyes for adjusting the pH there are preferably used mineral acids, in particular HCl, inorganic bases, in particular NaOH or KOH, or organic acids, in particular lactic acid.

Sorbitan caprylate can preferably be used as the activity enhancer.

Within the scope of a preferred embodiment, the composition is free of alkyl sulfates and/or alkyl ether sulfates. Free means that the composition contains less than 3% by weight, based on the total amount of the composition, preferably less than 0.5% by weight and in particular no alkyl sulfates and/or alkyl ether sulfates.

Within the scope of a preferred embodiment, the composition according to the invention is a cosmetic, dermatological or pharmaceutical composition.

The invention further provides the use of the composition according to the invention as a shampoo, facial cleanser, liquid cleanser or shower gel.

The invention further provides the use of the composition according to the invention for the treatment or care of the skin.

The invention further provides the use of the composition according to the invention for the treatment or care of the hair.

The invention will be explained in greater detail by the following examples.

### PREPARATION EXAMPLES P1 AND P2

The N-acyl-N-methyl-glucamines described in the following were prepared according to EP 0 550 637 from the corresponding fatty acid methyl esters and N-methylglucamine in the presence of 1,2-propylene glycol as solvent and were obtained in the form of a solid consisting of active substance and 1,2-propylene glycol.

TABLE 1

| | | Active | | |
|---|---|---|---|---|
| Preparation | Methyl | substance | 1,2-Propylene | Melting |
| Example | ester | (%) | glycol (%) | point ° C. |
| P1 | C12/18 | 88 | 12 | 80 |
| P2 | C16/18 | 80 | 20 | 68 |

C12/C18 means that the methyl ester consists of a mixture of lauric acid methyl ester (C₁₂-acyl radical), myristic acid methyl ester (C₁₄-acyl radical), palmitic acid methyl ester (C₁₆-acyl radical), stearic acid methyl ester (C₁₈-acyl radical) and oleic acid methyl ester (C₁₈-acyl radical) (ratio 64:21:2:3:10). C16/18 means that the methyl ester consists of a mixture of palmitic acid methyl ester (C₁₆-acyl radical) and stearic acid methyl ester (C₁₈-acyl radical) (ratio 30:70).

The viscosities are measured at 20 revolutions/minute and 20° C. using a Brookfield viscometer model DV II, the spindles from the RV spindle set. Spindles 1 to 7 from the RV spindle set are used. Under these measuring conditions, spindle 1 is chosen for viscosities of not more than 500 mPa·s, spindle 2 for viscosities of not more than 1000 mPa·s, spindle 3 for viscosities of not more than 5000 mPa·s, spindle 4 for viscosities of not more than 10,000 mPa·s, spindle 5 for viscosities of not more than 20,000 mPa·s, spindle 6 for viscosities of not more than 50,000 mPa·s and spindle 7 for viscosities of not more than 200,000 mPa·s.

In the following test formulation, oil-containing shower creams with and without added N-acyl-N-methylglucamine according to Preparation Example P1 and P2 were tested in comparison to formulations without N-acyl-N-methylglucamine. The amounts used relate to the active content of the components, that is to say 2.0% of Preparation Example P1 correspond to an amount of reaction product that is actually used of 2.27%.

It will be seen that stable emulsions are obtained when, in addition to oily substances and a fatty acid, amino acid surfactant (sodium cocoyl glycinate) and N-acyl-methylglucamine are present at the same time (formulations according to the invention Examples 1-4).

| | Formulation Example Component (%) | | | | | | | | | | | | | |
|---|---|---|---|---|---|---|---|---|---|---|---|---|---|---|
| | C1 | C2 | C3 | C4 | C5 | C6 | 1 | 2 | 3 | 4 | C7 | C8 | C9 | C10 |
| Water | ad | ad | ad | ad | ad | ad | ad | ad | ad | ad | ad | ad | ad | ad |
| | 100 | 100 | 100 | 100 | 100 | 100 | 100 | 100 | 100 | 100 | 100 | 100 | 100 | 100 |
| Sodium lauryl ether | 5.0 | 6.0 | 5.0 | 6.0 | 5.0 | 6.0 | 5.0 | 6.0 | 5.0 | 6.0 | 7.0 | 8.0 | 7.0 | 8.0 |
| sulfate (2 EO) | | | | | | | | | | | | | | |
| Sodium cocoyl glycinate | 2.0 | 2.0 | 0.0 | 0.0 | 0.0 | 0.0 | 2.0 | 2.0 | 2.0 | 2.0 | 0.0 | 0.0 | 0.0 | 0.0 |
| Preparation Example P1 | 0.0 | 0.0 | 2.0 | 2.0 | 0.0 | 0.0 | 2.0 | 2.0 | 0.0 | 0.0 | 2.0 | 2.0 | 0.0 | 0.0 |
| Preparation Example P2 | 0.0 | 0.0 | 0.0 | 0.0 | 2.0 | 2.0 | 0.0 | 0.0 | 2.0 | 2.0 | 0.0 | 0.0 | 2.0 | 2.0 |
| Lauric acid | 2.0 | 2.0 | 2.0 | 2.0 | 2.0 | 2.0 | 2.0 | 2.0 | 2.0 | 2.0 | 2.0 | 2.0 | 2.0 | 2.0 |
| Sunflower oil | 1.5 | 1.0 | 1.5 | 1.0 | 1.5 | 1.0 | 1.5 | 1.0 | 1.5 | 1.0 | 1.5 | 1.0 | 1.5 | 1.0 |
| Perfume water lily | 1.0 | 1.0 | 1.0 | 1.0 | 1.0 | 1.0 | 1.0 | 1.0 | 1.0 | 1.0 | 1.0 | 1.0 | 1.0 | 1.0 |
| Sodium cocoyl | 1.2 | 1.2 | 1.2 | 1.2 | 1.2 | 1.2 | 1.2 | 1.2 | 1.2 | 1.2 | 1.2 | 1.2 | 1.2 | 1.2 |
| isethionate | | | | | | | | | | | | | | |
| Glycerol | 1.4 | 1.0 | 1.4 | 1.0 | 1.4 | 1.0 | 1.4 | 1.0 | 1.4 | 1.0 | 1.4 | 1.0 | 1.4 | 1.0 |
| Sodium chloride | 0.5 | 0.5 | 0.5 | 0.5 | 0.5 | 0.5 | 0.5 | 0.5 | 0.5 | 0.5 | 0.5 | 0.5 | 0.5 | 0.5 |
| Stearic acid | 1.0 | 1.0 | 1.0 | 1.0 | 1.0 | 1.0 | 1.0 | 1.0 | 1.0 | 1.0 | 1.0 | 1.0 | 1.0 | 1.0 |
| Cocamidopropyl betaine | 0.5 | 0.2 | 0.5 | 0.2 | 0.5 | 0.2 | 0.5 | 0.2 | 0.5 | 0.2 | 0.5 | 0.2 | 0.5 | 0.2 |
| Citric acid to | pH | pH | pH | pH | pH | pH | pH | pH | pH | pH | pH | pH | pH | pH |
| | 6.2 | 6.2 | 6.2 | 6.2 | 6.2 | 6.2 | 6.2 | 6.2 | 6.2 | 6.2 | 6.2 | 6.2 | 6.2 | 6.2 |
| DMDM hydantoin | 0.2 | 0.2 | 0.2 | 0.2 | 0.2 | 0.2 | 0.2 | 0.2 | 0.2 | 0.2 | 0.2 | 0.2 | 0.2 | 0.2 |
| Stability after 1 day | no | no | no | no | no | no | yes | yes | yes | yes | no | no | no | no |
| Stability after 1 week | no | no | no | no | no | no | yes | yes | yes | yes | no | no | no | no |
| Viscosity 4° C. | — | — | — | — | — | — | 258 | 194 | 228 | 218 | — | — | — | — |
| (mPas) | | | | | | | | | | | | | | |
| Viscosity 20° C. | — | — | — | — | — | — | 296 | 140 | 166 | 102 | — | — | — | — |
| (mPas) | | | | | | | | | | | | | | |
| Viscosity 40° C. | — | — | — | — | — | — | 282 | 110 | 154 | 78 | — | — | — | — |
| (mPas) | | | | | | | | | | | | | | |

The tests which were performed also show that a stable emulsion is obtained when glycinate surfactant and glucamide surfactant are present in the composition at the same time. If, on the other hand, the glycinate surfactant in the presence of glucamides is replaced by sodium lauryl ether sulfate, stable emulsions are not found.

In the further examples, an oil-containing shower gel was thickened with hydroxypropyl starch phosphate in order to obtain good rheological behavior upon application.

| Formulation Example | | |
|---|---|---|
| Component (%) | 5 | C11 |
| Water | ad 100 | ad 100 |
| Na laureth sulfate (2 EO) | 3 | 3 |
| Na cocoyl glycinate | 2 | 2 |
| Hydroxypropyl starch phosphate | 2 | 2 |
| Preparation Example P1 | 2 | 0 |
| Lauric acid | 1.6 | 1.6 |
| Sunflower oil | 1.2 | 1.2 |
| Perfume water lily | 1 | 1 |
| Na cocoyl sethionate | 1 | 1 |
| Glycerol | 1 | 1 |
| NaCl | 1 | 1 |
| Stearic acid | 2 | 2 |
| Cocamidopropyl betaine | 3 | 3 |
| Citric acid | pH 6.2 | pH 6.2 |
| DMDM hydantoin | 0.2 | 0.2 |
| Viscosity at 4° C. (mPas) | 3000 | 3020 |
| Viscosity at 20° C. (mPas) | 4165 | 2410 |
| Viscosity at 40° C. (mPas) | 4650 | 2660 |

As can be seen from Comparative Example 11 and Example 5, the addition of component B (Preparation Example 1) with a constant thickener concentration (hydroxypropyl starch phosphate) leads to a higher viscosity at 20° C. and 40° C. This is a desirable effect because, on the one hand, less polymer is needed to achieve the target viscosity and, on the other hand, the better viscosity profile at 40° C. means that more storage stable emulsions are obtained.

1. A composition comprising:
(A) at least one N-acyl-amino acid surfactant as component A, wherein component A consists of at least one C₈-C₂₂-acylated amino acid and is selected from the group consisting of acyl glycinate, acyl aspartate, acyl glutamate, acyl sarcosinate, their salts and mixtures thereof,
(B) at least one N-methyl-N-acylglucamine as component B,
(C) at least one C₈-C₂₂-fatty acid or a fatty acid salt as component C,
(D) at least one oily substance as component D,
(E) optionally at least one acyl isethionate as component E,
(F) optionally at least one anionic, sulfonated surfactant as component F,
(G) optionally at least one betaine surfactant as component G,
(H) optionally at least one additive as component H, and water.

2. The composition as claimed in claim 1, comprising:
(A) at least one N-acyl-amino acid surfactant as component A, wherein component A consists of at least one C₈-C₂₂-acylated amino acid and is selected from the group consisting of acyl glycinate, acyl aspartate, acyl glutamate, acyl sarcosinate, their salts and mixtures thereof,
(B) at least one N-methyl-N-acylglucamine as component B,
(C) at least one C₁₂-C₂₂-fatty acid or fatty acid salt as component C,
(D) at least one oily substance as component D,
(E) at least one acyl isethionate as component E,
(F) optionally at least one anionic, sulfonated surfactant as component F,
(G) optionally at least one betaine surfactant as component G,
(H) optionally at least one additive as component H, and water.

3. The composition as claimed in claim 1, consisting of:
(A) from 1 to 10% by weight of component A,
(B) from 0.5 to 10% by weight of component B,
(C) from 0.5 to 5.0% by weight of component C,
(D) from 0.5 to 8.0% by weight of component D,
(E) from 0 to 5.0% by weight of component E,
(F) from 0 to 10% by weight of component F,
(G) from 0 to 5% by weight of component G,
(H) from 0 to 15% by weight of one or more additives H, and
(I) from 74 to 98% by weight water,
wherein the sum of components A to I is 100% by weight.

4. The composition as claimed in claim 1, consisting of:
(A) from 1 to 10% by weight of component A,
(B) from 0.5 to 10% by weight of component B,
(C) from 0.5 to 5.0% by weight of component C,
(D) from 0.5 to 8.0% by weight of component D,
(E) from 0.5 to 5.0% by weight of component E,
(F) from 0 to 10% by weight of component F,
(G) from 0 to 5% by weight of component G,
(H) from 0 to 15% by weight of one or more additives H, and
(I) from 74 to 97.5% by weight water,
wherein the sum of components A to I is 100% by weight.

5. The composition as claimed in claim 1, consisting of:
(A) from 1 to 6.0% by weight of component A,
(B) from 1 to 5.0% by weight of component B,
(C) from 1 to 4.0% by weight of component C,
(D) from 1 to 5.0% by weight of component D,
(E) from 0.5 to 2.0% by weight of component E,
(F) from 1 to 5% by weight of component F,
(G) from 0.5 to 3% by weight of component G,
(H) from 5 to 10% by weight of one or more additives H,
(I) from 40 to 89% by weight water,
wherein the sum of components A to I is 100% by weight.

6. The composition as claimed in claim 1, wherein component A is selected from the group consisting of acyl glycinate, its salts and mixtures thereof.

7. The composition as claimed in claim 1, wherein component B consists of at least one C₈-C₂₂ N-methyl-Nacylglucamine.

8. The composition as claimed in claim 7, wherein component B consists of at least one C₁₂-C₁₈ N-methyl-N-acylg lucam in e.

9. The composition as claimed in claim 1, wherein component C consists of lauric acid, palmitic acid, stearic acid, their salts or mixtures thereof.

10. The composition as claimed in claim 1, wherein component D consists of an oily substance or of a mixture of oily substances selected from the group consisting of the triglyceride oils, the ester oils, the hydrocarbon oils and silicone oils.

11. The composition as claimed in claim 10, wherein component D consists of an oily substance or of a mixture of oily substances selected from the group consisting of the triglyceride oils.

12. The composition as claimed in claim 1, component E is present and consists of sodium lauroyl isethionate, sodium cocoyl isethionate or a mixture thereof.

13. The composition as claimed in claim 1, wherein component F is present and consists of sodium lauryl ether sulfate, sodium myreth sulfate, sodium lauryl sulfate, sodium cocoyl sulfate or mixtures thereof.

14. The composition as claimed in claim 1, wherein the composition is free of component F.

15. The composition as claimed in claim 1, wherein component G is present and consists of cocamidopropyl betaine, lauramidopropyl betaine, coco-betaine or mixtures thereof.

16. The composition as claimed in claim 1, wherein at least one additive (H) are present, and is selected from the group consisting of preservatives, fragrances, dyes, further surfactants which do not fall under the definition of components A to G, cationic polymers, film-forming agents, thickeners and gelling agents, superfatting agents, antimicrobial and biogenic active ingredients, moisture-donating agents, stabilizers, acids, lyes and activity enhancers.

17. The composition as claimed in claim 1, wherein the sum of components A to H is from 10 to 30% by weight.

18. A cosmetic, dermatological or pharmaceutical composition comprisinq at least one composition according to claim 1.

19. A shampoo, facial cleanser, shower gel or shower cream comprising at least one composition according to claim 1.

20. A treatment or care of the skin, of the hair or of the skin and the hair comprising at least one composition according to claim 1.
